(19) United States
(12) Patent Application Publication
(10) Pub. No.: US 2004/0116950 A1
(43) Pub. Date: Jun. 17, 2004

(54) Instrument and method for creating an intraocular incision

(75) Inventor: Maya Eibschitz-Tsimhoni, Ann Arbor, MI (US)

Correspondence Address: BROOKS KUSHMAN P.C., 1000 TOWN CENTER, TWENTY-SECOND FLOOR, SOUTHFIELD, MI, 48075, US

(73) Assignee: The Regents of the University of Michigan, Ann Arbor, MI

(21) Appl. No.: 10/681,695

(22) Filed: Oct. 7, 2003

Related U.S. Application Data
(63) Continuation-in-part of application No. 09/724,252, filed on Nov. 28, 2000, now Pat. No. 6,629,980.

(51) Int. Cl. A61B 17/32

(52) U.S. Cl. 606/167; 606/166

(57) ABSTRACT

An instrument and method are provided for incising an intraocular tissue, such as the anterior capsule of the eye, which requires only a small wound cut in the corneal or scleral tissue. The instrument includes a handle and a head portion attached to the handle, the head portion including spaced first and second ends which define an opening therebetween. The head portion further includes a cutting edge which is operable to engage the intraocular tissue and penetrate at least partially therethrough to create a first incision therein. Creating a second incision in the intraocular tissue adjacent the first incision results in a continuous tissue flap incised in the intraocular tissue.

### CROSS-REFERENCE TO RELATED APPLICATIONS

This application is a continuation-in-part of U.S. application Ser. No. 09/724,252 filed Nov. 28, 2000, now U.S. Pat. No. 6,629,980.

### TECHNICAL FIELD

This invention relates to an instrument and method for creating an incision in an intraocular tissue, such as the anterior capsule of the eye.

### BACKGROUND ART

An individual's vision involves the eye forming an image of an object and sending that image to the sensory centers of the brain. An object will reflect light through the cornea, the aqueous humor, the pupil, the lens, and the vitreous humor of the eye, wherein the reflective light is focused by the lens onto the retina (see FIG. 1). The nerve fibers within the retina collectively leave the eye in the optic nerve and enter the brain where the visual signals are processed.

Among other factors, therefore, the quality of vision depends upon the transparency of the lens. An opacity of the lens, commonly known as cataract, may prevent a clear image from forming on the retina. The lens is encapsulated by a cellophane-like membrane covering its anterior and posterior surfaces, wherein the capsule is retained in position chiefly by suspensory ligaments termed zonules. Cataracts may be age-related, congenital or result from trauma, disease or medications, and are generally treated by performing extracapsular cataract extraction. In this procedure, an opening is provided in the anterior lens capsule through which instrumentation can enter and the opaque lens is removed and replaced by an artificial intraocular lens.

Each year, approximately 1.3 million cataract surgeries are performed in the United States, and several methods have been utilized for opening the anterior lens capsule to gain access to the lens nucleus and cortical material. Currently, the two most popular techniques for anterior capsulotomy are the “can-opener” technique and capsulorrhexis. In can-opener capsulotomy, a cystotome, knife, or needle is inserted through a small incision in the sclera or peripheral cornea and small connecting tears are made in the anterior lens capsule in a circular pattern. When a complete circle has been made by connecting the tears, a circular piece of the anterior capsule is then grasped with forceps and torn away along the perforations. Unfortunately, when opening the capsule with numerous small capsular tears, the small tags which remain become a focal area of least resistance and can lead to tears which extend radially and posteriorly to the posterior capsule. The detrimental result is a loss of structural stability of the capsule and an increased likelihood of vitreous entry into the anterior chamber.

Capsulorrhexis denotes a circular central opening in the anterior capsule. This continuous opening eliminates the residual tags common with the can-opening technique described above. In capsulorrhexis, a capsular incision is made with a cystotome, and this incision is coaxed to form a circular shape by pushing the leading edge of the freshly tearing capsule with the cystotome in a non-cutting fashion or by grasping the leading edge with forceps. This procedure is quite difficult to control by the surgeon. The tearing motion can lead to an undesirable tear toward the equator and the posterior capsule, and the opening size is hard to control. As such, capsulorrhexis requires a significant amount of skill, experience, and learning time to consistently obtain successful results.

As described above, opening the anterior capsule via an anterior capsulotomy is a very delicate procedure and is widely considered to be one of the most difficult steps in a cataract surgery. A poorly performed anterior capsulotomy significantly increases the difficulty in performing the subsequent surgical steps and the probability of operative complications. Complications resulting from a poor capsulotomy include zonular stress with subsequent breakage of the posterior capsule, vitreous loss, and large capsular tags preventing efficient lens removal. A poor capsulotomy also prevents placement of an intraocular lens in the capsular bag due to ill-defined capsular structures. Such complications are unfortunately frequent. An unsuccessful capsulotomy increases the risk of intraoperative complications such as vitreous loss and inability to implant a posterior chamber intraocular lens. The operative time and patient discomfort are increased, along with the risk of postoperative complications with decreased final best-corrected visual acuity results.

Furthermore, with either of the above-described techniques for anterior capsulotomy, the capsular opening's size or position is often not ideal. The location, size, and configuration of the incision have important consequences. For example, a small capsular opening can impair the safe removal of the lens nucleus and cortex and prevent proper intraocular lens insertion into the lens capsule. In addition, the excessive stress placed on the lens capsule which result from having to work with a small or eccentric capsular opening puts the eye at risk for zonular and capsular breakage.

Certain devices have been proposed to overcome the problems associated with conventional anterior capsulotomy techniques. For example, U.S. Pat. No. 4,766,897 issued to Smirmaul, and U.S. Pat. Nos. 5,269,787 and 5,873,883 issued to Cozean Jr. et al. each disclose instruments which include circular cutting members for incising the anterior capsule. However, use of such devices in small incision cataract surgery is limited due to their size. More particularly, the anterior lens capsule of the eye is shielded by the cornea and sclera, such that a passage wound must be cut in the corneal or scleral tissue before any surgical apparatus can reach the anterior capsule. It is desirable to have the width of the passage wound cut on the tissue as small as possible, preferably on the order of 2.4 to 2.7 mm in width. A small wound decreases the necessary surgical closing procedures, promote rapid healing, minimizes astigmatism, reduces potential infections, and offers rapid visual rehabilitation. Therefore, the instrumentation employed in cataract surgery should be capable of passing through a small wound.

In an attempt to meet this size requirement, alternative surgical devices have been proposed. For example, U.S. Pat. No. 5,135,530 issued to Lehmer discloses a deformable circular cutting ring which is provided between the two forward positions of two elongated arms. The arms crisscross each other and are hinged together, such that when the rearward portions of the two arms are squeezed toward each other, the forward positions of the two arms will move toward each other to compress the deformable circular cutting ring into a narrow elliptical shape. The overall width of the narrow elliptical shaped deformable circular cutting ring and the forward portions of the two elongated arms is small enough to be inserted into the anterior chamber of the eye through a small tissue wound of about 4 mm in width.

Similarly, U.S. Pat. No. 5,728,117 issued to Lash discloses a capsulorrhexis instrument that includes a flexible band having a cutting edge. The band is fixed to a plunger, and is retractable within a tube and extendable into a position projecting out of the tube. While in its retracted position within the tube, the band assumes a narrow elliptical shape. However, when the flexible band is in its extended position outside of the tube, such as inside the eye, it deforms into a circular shape for incising intraocular tissue.

While these deformable devices provide one solution to the size constraint imposed by a small tissue wound, the devices suffer from the disadvantages of being overly complex as well as costly to manufacture and maintain. Therefore, a need exists for an improved instrument and method for creating an intraocular incision within the constraints of small incision cataract surgery.

### DISCLOSURE OF INVENTION

Therefore, it is an object according to the present invention to provide an instrument and method for incising an intraocular tissue which is capable of creating an incision of suitable size for lens removal, yet which requires only a small entry wound cut on the corneal or scleral tissue.

It is another object according to the present invention to provide an instrument and method for incising an intraocular tissue which allows for precise control over the location and size of the resulting incision.

It is yet another object according to the present invention to provide an instrument and method for incising an intraocular tissue which is easy to operate and simple and cost-efficient to manufacture.

Accordingly, an instrument is provided for incising an intraocular tissue, such as the anterior capsule of the eye. The instrument includes a handle and a head portion attached to the handle. The head portion has a cutting edge, wherein the head portion defines a plane and the cutting edge extends from the head portion in a direction substantially transverse to the plane. The head portion includes a main portion having a first radius and at least one extension portion continuous with the main portion and having a second radius different from the first radius. The head portion further includes spaced first and second ends which define an opening therebetween. The cutting edge is operable to engage the intraocular tissue and penetrate at least partially therethrough to create an incision in the intraocular tissue.

In a preferred embodiment, the second radius is smaller than the first radius, with the first radius between about 1.0 to 4.0 mm, and the second radius between about 0.25 to 2.0 mm. The handle can be connected to the head portion at an end of the extension portion, at an intermediate point of the extension portion or the main portion, or at a junction of the main portion and the extension portion.

In one embodiment, the head portion is generally C-shaped and the extension portion comprises a proximal leg extending from a first end of the main portion and a distal leg extending from a second end of the main portion. The head portion can also include a wavy configuration, and can include a tongue extending from a distal end of the head portion. Additionally, the head portion can include a second cutting edge opposing the cutting edge, and either cutting edge may be serrated. Preferably, the handle includes a generally horizontal portion extending along the plane of the head portion and an angled portion extending at an angle relative to the horizontal portion. Still further, the handle is preferably connected to the head portion at a location greater than about 0.3 mm above the cutting edge.

In further accordance with the present invention, an instrument for incising an intraocular tissue is provided which includes a handle and a generally C-shaped head portion. The head portion has a cutting edge, where the head portion defines a plane and the cutting edge extends from the head portion in a direction substantially transverse to the plane. The head portion includes a generally semicircular central portion having a first radius, the central portion having first and second ends, and proximal and distal legs continuous with the first and second ends of the central portion, respectively, the legs each having a second radius different from the first radius. In a preferred embodiment, the handle is connected to the head portion at a location inward from an end of the proximal leg. The cutting edge is operable to engage the intraocular tissue and penetrate at least partially therethrough to create a curvilinear incision in the intraocular tissue.

According to another embodiment of the present invention, an instrument for incising an intraocular tissue includes a handle and a head portion having a cutting edge, the head portion defining a plane and the cutting edge extending from the head portion in a direction substantially transverse to the plane. The head portion includes a first portion having a first radius and a second portion continuous with the first portion and having a second radius different from the first radius. The handle can be connected to an end of the second portion, or to an intermediate point of the second portion or the first portion. The cutting edge is operable to engage the intraocular tissue and penetrate at least partially therethrough to create an incision in the intraocular tissue.

In further accordance with the present invention, a surgical kit for incising an intraocular tissue is provided. The kit includes a first instrument having a handle and a head portion attached to the handle, the first instrument head portion including a main portion having a first radius and at least one extension portion continuous with the main portion and having a second radius different from the first radius. The first instrument head portion has spaced first and second ends which define an opening therebetween having a first orientation, where the first instrument head portion includes a cutting edge such that the head portion is operable to engage the intraocular and penetrate at least partially therethrough to create a first incision in the intraocular tissue. The kit further includes a second instrument having a handle and a head portion attached to the handle, the second instrument head portion including a main portion having a first radius and at least one extension portion continuous with the main portion and having a second radius different from the first radius. The second instrument head portion has spaced first and second ends which define an opening therebetween having a second orientation, where the second instrument head portion includes a cutting edge such that the head portion is operable to engage the intraocular tissue and penetrate at least partially therethrough to create a second incision in the intraocular tissue. Creating adjacent first and second incisions results in a continuous tissue flap incised in the intraocular tissue.

Correspondingly, a method is provided for incising an intraocular tissue. The method includes creating a wound in one of a corneal region and a scleral region of the eye. Next, the method includes inserting an instrument through the wound and into proximity with the intraocular tissue, where the instrument includes a handle and a head portion attached to the handle having a cutting edge. The head portion includes a main portion having a first radius and at least one extension portion continuous with the main portion and having a second radius different from the first radius, the head portion including spaced first and second ends which define an opening therebetween. The method further includes engaging the intraocular tissue with the cutting edge and penetrating at least partially through the intraocular tissue in order to create an incision therein.

In greater particularity, a method is provided for incising an intraocular tissue which includes creating a wound in one of a corneal region and a scleral region of the eye, and inserting a first instrument through the wound and into proximity with the intraocular tissue. The first instrument includes a handle and a head portion attached to the handle, the head portion including a main portion having a first radius and at least one extension portion continuous with the main portion and having a second radius different from the first radius, the first instrument head portion including a cutting edge having a first orientation. Next, the method includes engaging the anterior capsule with the first instrument cutting edge and penetrating at least partially through the intraocular tissue in order to create a first incision therein. The method further includes inserting a second instrument through the wound and into proximity with the intraocular tissue. The second instrument includes a handle and a head portion attached to the handle, the head portion including a main portion having a first radius and at least one extension portion continuous with the main portion and having a second radius different from the first radius, the second instrument head portion including a cutting edge having a second orientation which opposes the first orientation. Still further, the method includes engaging the intraocular tissue with the second instrument cutting edge and penetrating at least partially through the intraocular tissue in order to create a second incision therein adjacent the first incision, thereby resulting in a continuous tissue flap incised in the intraocular tissue.

In one embodiment, the first and second instruments each include a generally C-shaped head portion including a generally semicircular central portion having a first radius, the central portion having first and second ends, and proximal and distal legs continuous with the first and second ends of the central portion, respectively. The legs each have a second radius smaller than the first radius. With this configuration, imprecise alignment of the first and second incisions can still result in an overlap of the first and second incisions and a continuous tissue flap.

In another embodiment, the first and second instruments each include a head portion including a first portion having a first radius and a second portion continuous with the first portion and having a second radius smaller than the first radius. With this configuration, adjacent first and second incisions can create an enlarged end of the tissue flap proximal to the wound.

An alternative method for incising an intraocular tissue according to the present invention includes creating a wound in one of a corneal or scleral region of the eye, and inserting an instrument through the wound and into proximity with the intraocular tissue. The instrument includes a handle and a head portion attached to the handle, where the head portion has first and second opposed cutting edges. The method further includes engaging the intraocular tissue with the first cutting edge and penetrating at least partially through the intraocular tissue in order to create a first incision therein. Next, the method includes rotating the instrument approximately 180° about a longitudinal axis of the handle. Still further, the method includes engaging the intraocular tissue with the second cutting edge and penetrating at least partially through the intraocular tissue in order to create a second incision therein adjacent the first incision, thereby resulting in a continuous tissue flap incised in the intraocular tissue.

Still further, an instrument for incising an intraocular tissue is provided including a handle and a curvilinear head portion attached to the handle. The head portion includes a cutting edge, where the head portion defines a plane and the cutting edge extends from the head portion in a direction substantially transverse to the plane. The head portion further includes spaced first and second ends which define an opening therebetween, and a tongue extends outwardly from a distal end of the head portion along the plane of the head portion. The cutting edge is operable to engage the intraocular tissue and penetrate at least partially therethrough to create an incision in the intraocular tissue.

The above objects and other objects, features, and advantages of the present invention are readily apparent from the following detailed description of the best mode for carrying out the invention when taken in connection with the accompanying drawings.

### BRIEF DESCRIPTION OF DRAWINGS

FIG. 1 is a cross-sectional view illustrating the human eye;

FIG. 2a is a side elevational view of an instrument for incising an intraocular tissue in accordance with the present invention, wherein one cutting edge is provided and the head portion has a right-facing orientation;

FIG. 2b is a cross-sectional view of the head portion of FIG. 2a taken along line 2b-2b;

FIG. 3 is a top plan view of the instrument of FIG. 2a;

FIG. 4 is a top plan view of an instrument in accordance with the present invention wherein the head portion has a left-facing orientation;

FIG. 5a is a perspective view of an alternative embodiment of the instrument of the present invention, wherein two cutting edges are provided;

FIG. 5b is a cross-sectional view of the head portion of FIG. 5b taken along line 5b-5b;

FIG. 6a is a side elevational view of the instrument of FIG. 2a wherein the head portion is attached to the handle at an angle a relative to the longitudinal axis of the handle;

FIG. 6b is a top plan view of the instrument of FIG. 2a wherein the head portion is attached to the handle at an angle β relative to the longitudinal axis of the handle;

FIG. 7 is a cross-sectional view of the eye illustrating the position of the instrument relative to the eye once the instrument is inserted through the wound cut in the corneal or scleral tissue and brought into proximity with the anterior lens capsule;

FIG. 8 is a top plan view illustrating the anterior capsule prior to entry of a right-facing instrument in accordance with the present invention through the wound;

FIG. 9 is a top plan view illustrating the creation of a first generally semicircular incision in the anterior capsule using the instrument of FIG.8;

FIG. 10 is a top plan view illustrating the creation of a second generally semicircular incision in the anterior capsule adjacent the first incision using a left-facing instrument in accordance with the present invention;

FIG. 11 is a top plan view illustrating removing the instrument of FIG. 10 through the wound, wherein a generally circular tissue flap in the anterior capsule has been formed by creating the first and second incisions;

FIGS. 12a and 12b are top plan views of another alternative embodiment of the instrument according to the present invention having right-facing and left-facing orientations, respectively;

FIG. 13a is a fragmentary, enlarged side elevational view of the head portion of the instrument of FIG. 12a;

FIG. 13b is a fragmentary, enlarged side elevational view of the head portion of the instrument of FIG. 12a showing an alternative cutting edge;

FIG. 14 is a top plan view of an alternative configuration of the instrument of FIG. 12a;

FIG. 15 is a top plan view of the instrument of FIG. 12a with the addition of a tongue provided on a distal end thereof;

FIGS. 16a and 16b are top plan views of another alternative embodiment of the instrument according to the present invention having right-facing and left-facing orientations, respectively;

FIG. 17 is a top plan view illustrating the creation of a first incision in the anterior capsule using the instrument of FIG. 12a;

FIG. 18 is a top plan view illustrating the creation of a second incision in the anterior capsule adjacent the first incision using the instrument of FIG. 12b;

FIG. 19a is a top plan view illustrating a generally circular tissue flap formed in the anterior capsule by the incisions of the instruments of FIGS. 12a and 12b;

FIG. 19b is a top plan view illustrating a tissue flap formed in the anterior capsule by the incisions of the instruments of FIGS. 12a and 12b when the first and second incisions are not precisely aligned;

FIG. 20 is a top plan view illustrating the creation of a first incision in the anterior capsule using the instrument of FIG. 16a;

FIG. 21 is a top plan view illustrating the creation of a second incision in the anterior capsule adjacent the first incision using the instrument of FIG. 16b; and

FIG. 22 is a top plan view illustrating a tissue flap formed in the anterior capsule by the incisions of the instruments of FIGS. 16a and 16b.

### BEST MODE FOR CARRYING OUT THE INVENTION

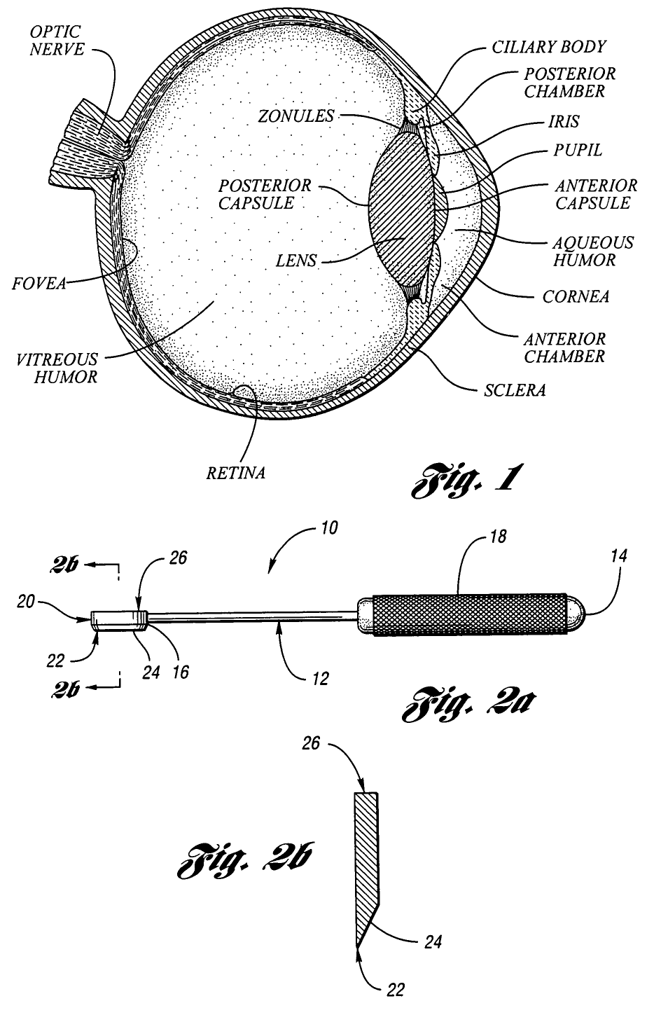

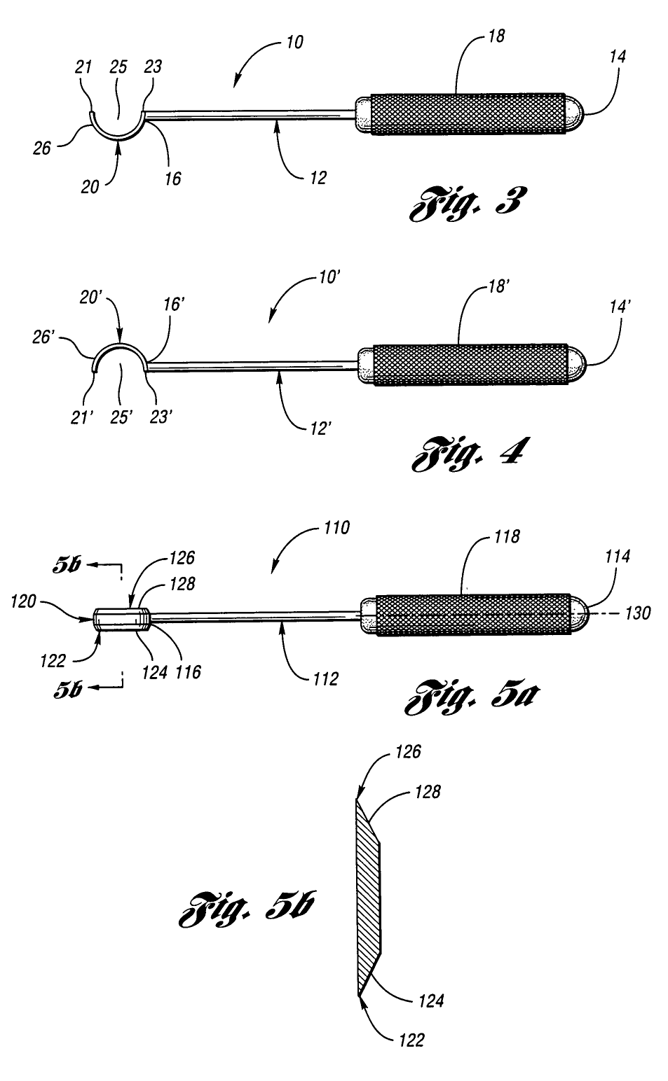

Referring first to FIGS. 2-3, an instrument, designated by reference numeral 10, for incising an intraocular tissue is shown constructed in accordance with the present invention. Instrument 10 is designed for use during an ophthalmic surgical procedure, such as in an anterior capsulotomy during cataract surgery, where an incision is required in an intraocular tissue. Instrument 10 is capable of being inserted through a small wound, which can be less than about 4 mm, cut in the corneal tissue or scleral tissue and into the anterior chamber of the eye (see FIG. 1 for eye anatomy). Instrument 10 then engages the intraocular tissue, namely the anterior lens capsule, in order to create an adequate sized incision in the anterior capsule so that the natural lens of the eye can be removed.

As shown in FIGS. 2-3, instrument 10 includes an elongated handle 12, which can be solid or hollow and is preferably of circular cross-section. Handle 12 can be of any length appropriate for the intended surgical procedure. For use in an anterior capsulotomy procedure, handle 12 is typically about 5 cm in length. Handle 12 has a proximal end 14 and a distal end 16, and preferably includes an enlarged portion 18 for grasping purposes. Handle 12 can also include an exterior grip surface, such as a knurled surface, to provide the operator with sufficient grip and manipulation of the instrument 10 during use.

Referring again to FIGS. 2 and 3, instrument 10 further includes a curvilinear head portion 20 attached to or near the distal end 16 of handle 12. Head portion 20 includes spaced first 21 and second 23 ends which define an opening 25 therebetween. Ends 21 and 23 can be rounded for safety, and can be constructed of a compliant material. In a preferred embodiment, head portion 20 is generally semicircular in shape, and can be constructed to be solid or hollow. Head portion 20 may be integrally formed with handle 12 or affixed thereto by welding or the like. In order to accommodate a small corneal/scleral wound on the order of 4 mm or less in length, head portion 20 is constructed to have a radius of between about 0.5 to 4 mm, and most preferably about 2 to 3 mm. Likewise, the width of head portion 20 is designed to be less than 3 mm, and most preferably about 1 mm. Of course, instrument 10 can be manufactured in many different dimensions such that the surgeon can select an instrument which is customized to the needs of a particular patient. For example, children's eyes are smaller than those of adults, such that head portion 20 should be designed to have a reduced width and radius when intended for use in pediatric surgical procedures.

As best shown in FIG. 2b, head portion 20 includes a bottom surface 22 having a cutting edge 24, where cutting edge 24 is operable to engage the intraocular tissue and penetrate at least partially through the tissue to create a curvilinear, or preferably generally semicircular, incision therein. More particularly, instrument 10 depicted in FIGS. 2 and 3 will create a generally semicircular incision with a first, right-facing orientation. Cutting edge 24 is preferably about 0.2 mm to 2.5 mm in height, most preferably about 0.5 mm. Head portion 20 includes a second surface 26 opposing the first surface 22 (FIGS. 2 and 3), wherein the second surface 26 is preferably smooth to avoid injury of intervening tissue while inserting and removing instrument 10 from the anterior chamber. Second surface 26 may also be visibly wider than cutting edge 24 to provide a visual cue to the surgeon as to the required orientation of instrument 10.

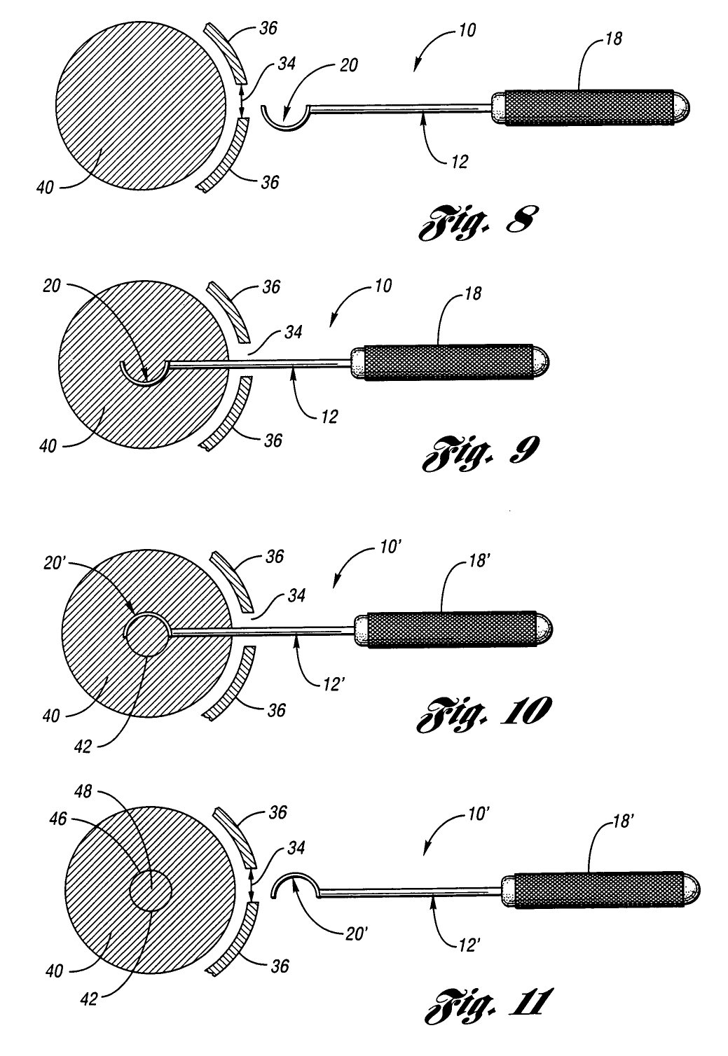

In practice, a continuous, preferably generally circular, incision is required in the anterior capsule in order to remove the natural lens material and also to insert an artificial intraocular lens. Therefore, as shown in FIG. 4, a second instrument 10′ is provided in accordance with the present invention, wherein the reference numerals for instrument 10′ correspond to those for instrument 10 except for the addition of a prime (′) designation. Instrument 10′ has a generally semicircular head portion 20′ with a second, left-facing orientation, such that instruments 10 and 10′ can be used successively to create the desired circular tissue flap. More particularly, instruments 10 and 10′ could be included in a surgical kit for incising an intraocular tissue, such as the anterior capsule. As described below with reference to FIGS. 7-11, cutting edge 24 of instrument 10 is operable to engage the anterior capsule and penetrate at least partially therethrough to create a first generally semicircular incision therein (FIG. 9). Then, cutting edge 24′ of instrument 10′, having an orientation which opposes that of instrument 10, is operable to engage the anterior capsule and penetrate at least partially therethrough to create a second generally semicircular incision therein (FIG. 10). Creating adjacent first and second incisions using instruments 10 and 10′ results in a continuous, generally circular tissue flap incised in the anterior capsule (FIG. 11).

Referring now to FIGS. 5a and 5b, an alternative instrument 110 can be provided, wherein reference numerals for instrument 110 correspond to those for instrument 10 except for the addition of a “1” prefix. Head portion 120 of instrument 110 has a second surface 126 which includes a cutting edge 128. Therefore, after cutting edge 124 can be used to make a first incision, instrument 110 is rotated approximately 180° about the handle longitudinal axis 130, and a second incision is made with cutting edge 128 adjacent the first incision in order to complete the circle.

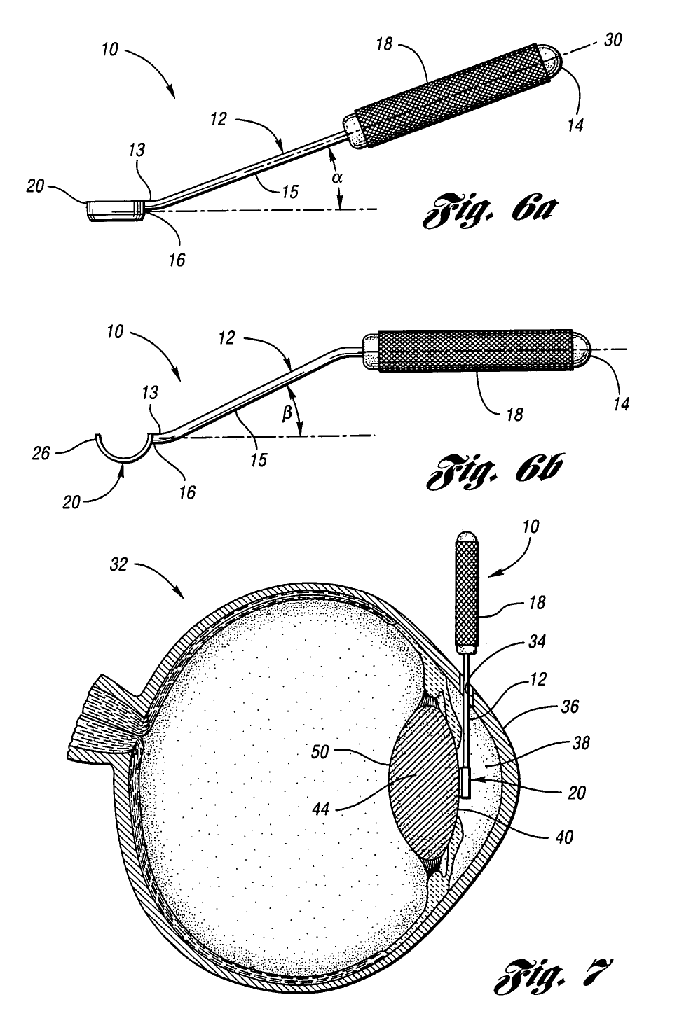

FIG. 6a illustrates an embodiment of instrument 10 wherein head portion 20 is attached to handle 12 at an angle α relative to longitudinal axis 30 of the handle 12. This configuration of instrument 10 may be advantageous when performing cataract surgery from above the forehead of the patient. Alternatively, FIG. 6b illustrate head portion attached to handle 12 at an angle β relative to longitudinal axis 30 of the handle 12, which may provide more hand clearance when using additional instruments adjacent instrument 10. Of course, instrument 10 can be configured to include both angles α and β if desired. Handle 12 can also include orientation marks (not shown) to help align the location of head portion 20 and its depth of insertion. Preferably, handle 12 includes a generally horizontal portion 13 extending along the plane of head portion 20 and an angled portion 15 extending at an angle relative to horizontal portion 13 in order to accommodate for the distance between the corneal/scleral wound and the incision site. Still further, handle 12 is preferably connected to head portion 20 at a location greater than about 1 mm above the cutting edge 24 to prevent engaging the iris with the handle 12 when the instrument 10 is pressed into the intraocular tissue. Handle 12 could also connect to instrument 10 on non-cutting edge 26, preferably at a 20 to 90° angle.

Instrument 10 can either be constructed entirely from a plastic, metallic, or other suitable material, or alternatively from a combination of such materials. More particularly, head portion 20 can be constructed from a medical grade of plastic or surgical steel, both of which can be sharpened to create cutting edge 24. As described below, the sharpness of cutting edge 24 need not be overly precise, since the operator can adjust the pressure applied to the intraocular tissue in order to achieve the particular tissue penetration desired. For any of the instruments described herein, the cutting edges need only to structurally weaken the tissue along the engagement site, after which the anterior capsule can be coaxed to tear along the engagement site and the tissue flap removed. Advantageously, due to its open curvilinear (e.g. semicircular) design, instrument 10 need not be constructed from flexible materials, as was required in certain prior art devices, in order to accommodate insertion through a small corneal/scleral wound. In fact, given its low cost and ease of manufacture, instrument 10 of the present invention is preferably disposable after use, providing obvious medical benefits with respect to contamination or the like. Alternatively, instrument 10 can be reused as long as its materials of construction are suitable for sterilization. As still another option, handle 12 could be designed to be reusable, and head portion 20 attached to handle 12 in such a way as to be removable and replaceable, similar to a conventional scalpel.

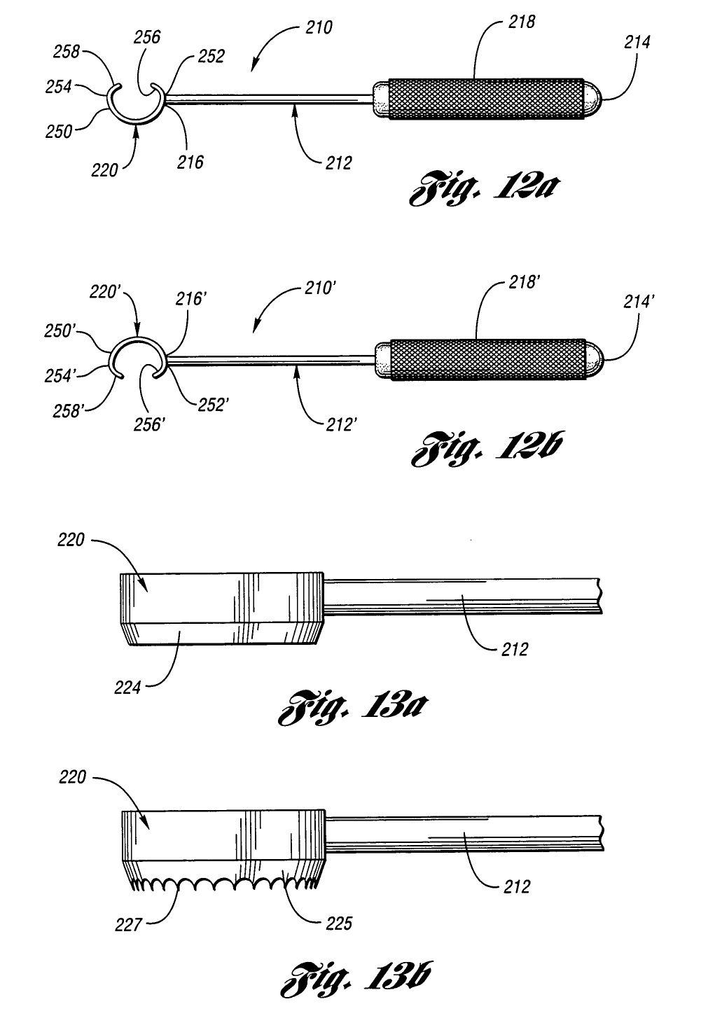

Referring next to FIGS. 12a and 12b, alternative instruments 210, 210′ are shown, wherein reference numerals for instruments 210, 210′ correspond to those for instruments 10, 10′ except for the addition of a “2” prefix. Except for the noted differences, it is understood that the foregoing description of instruments 10, 10′, 110 also applies to the alternative instrument embodiments described below. With reference to FIG. 12a, head portion 220 is generally C-shaped and includes a generally semicircular central portion 250 having a first radius, wherein the central portion 250 has first and second ends 252, 254. Proximal and distal legs 256, 258 are continuous with the first and second ends 252, 254 of the central portion 250, respectively, wherein the legs 256, 258 each have a second radius different from the first radius of the central portion 250. In a preferred embodiment, the first radius is larger than the second radius, with the first radius between about 1.0 to 4.0 mm, and the second radius between about 0.25 to 2.0 mm. Preferably, proximal and distal legs 256, 258 extend the semicircle of central portion 250 about 0.2 mm on each end 252, 254 thereof, and then each bend inwardly about 0.5 mm. Legs 256, 258 can be curved or straight segments, and preferably extend from central portion 250 at an angle between about 45 to 135°. Advantageously, proximal and distal legs 256, 258 ensure connection of the incisions created by instruments 210, 210′ even if the incisions are slightly shifted or rotated relative to each other as described below with reference to FIGS. 17-19.

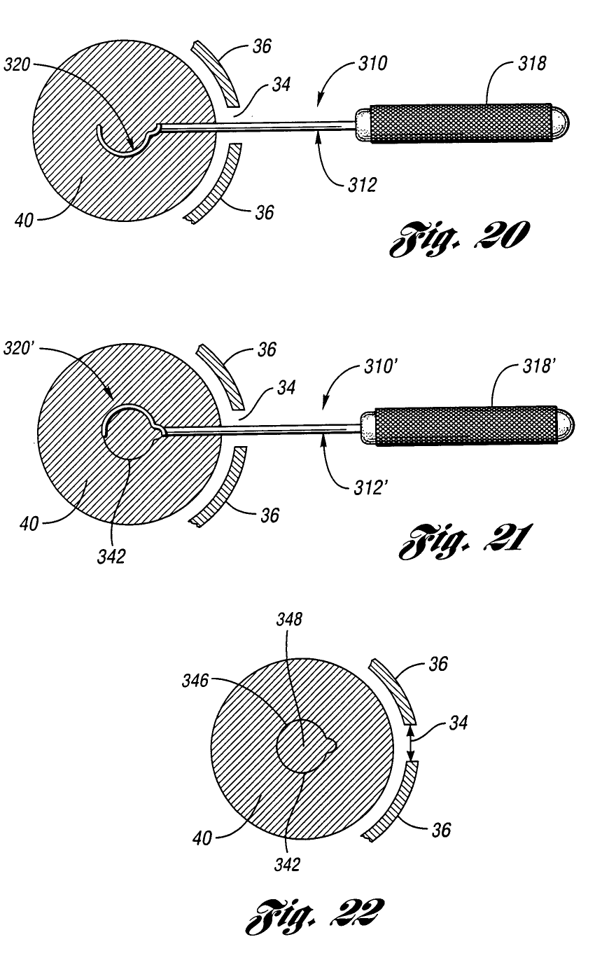

With continuing reference to FIGS. 12a and 12b, the distal end 216 of handle 212 is preferably connected to head portion 220 at a junction of central portion 250 and proximal leg 256 as depicted herein, or alternatively at an intermediate point of proximal leg 256 or central portion 250. This placement of handle distal end 216 at a location inward from an end of proximal leg 256 provides a guide for the surgeon when creating incisions in the intraocular tissue as described below with reference to FIGS. 19a and 19b.

In one embodiment, the cutting edge 224 of instrument 210 is generally straight as depicted in FIG. 13a, and is continuous along central portion 250 and proximal and distal legs 256, 258. As described above with reference to instrument 110, head portion 220 can include a second cutting edge (see FIG. 5a) opposing cutting edge 224. Furthermore, as shown in FIG. 13b, an alternative, serrated cutting edge 225 may be provided for any of the instrument embodiments disclosed herein. In a preferred embodiment, the spikes 227 are about 0.1 mm to 0.5 mm in height. Spikes 227 can have any spacing, preferably 0.01 to 2 mm, and the entire cutting edge 225 is sharp. Although a rounded-type serrated edge 225 is illustrated herein, a sawtooth or other like configuration is fully contemplated in accordance with the present invention. This type of cutting edge 225 facilitates initial penetration into the intraocular tissue and helps stabilize the instrument of the present invention during incision of the tissue. The spikes 227 of the cutting edge 225 enter first, making it easier to push the remainder of the cutting edge 225 further into the tissue for a complete cut. In an embodiment having two opposed cutting edges, both edges could be serrated, both edges could be straight, or one edge could be serrated and the other edge could be straight.

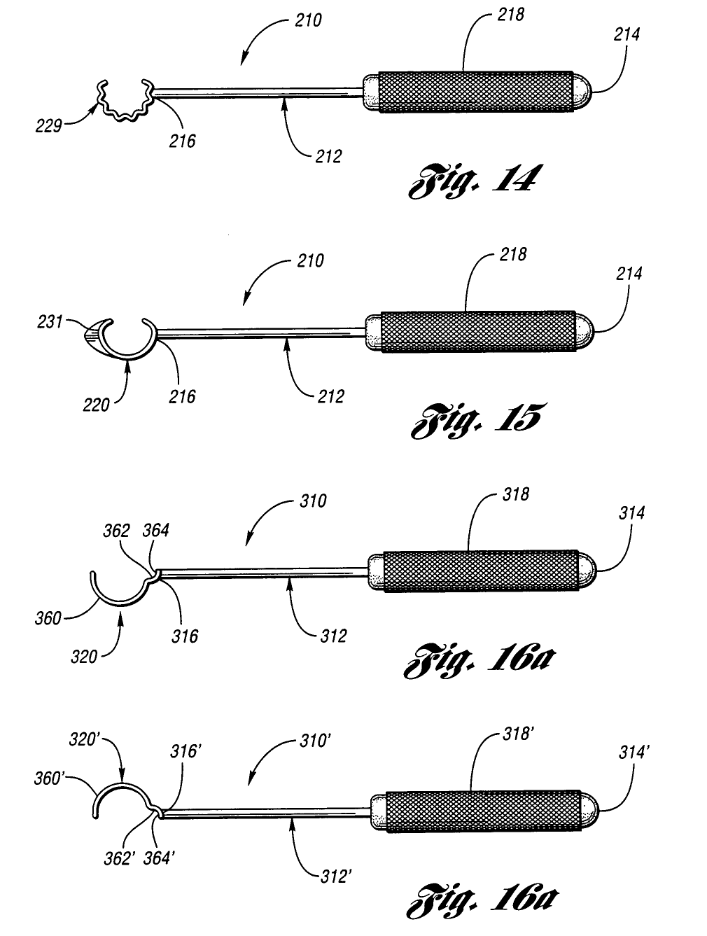

FIG. 14 illustrates another embodiment of instrument of the present invention wherein a wavy head portion 229 is provided. This wavy configuration increases the surface area of the instrument for a given radius, again facilitating incision of the intraocular tissue, and could be used for any of the instrument embodiments disclosed herein. In FIG. 15, a tongue 231 is provided extending from the distal end of head portion 220, preferably at least 0.3 mm above cutting edge 224. Tongue 231 can have any shape with dimensions more narrow than the wound, preferably 0.5 to 2.7 mm wide with a thickness of 0.1 to 1 mm. Tongue 231 has rounded, smooth edges and can be constructed from a different, preferably softer material than the head portion 220. Tongue 231 facilitates entrance of the instrument 210 through the wound and into the eye by holding the wound open as the remainder of the instrument 210 is introduced therein, thereby protecting the wound from cutting edge 224. Again, tongue 231 could be used with any of the instrument embodiments disclosed herein.

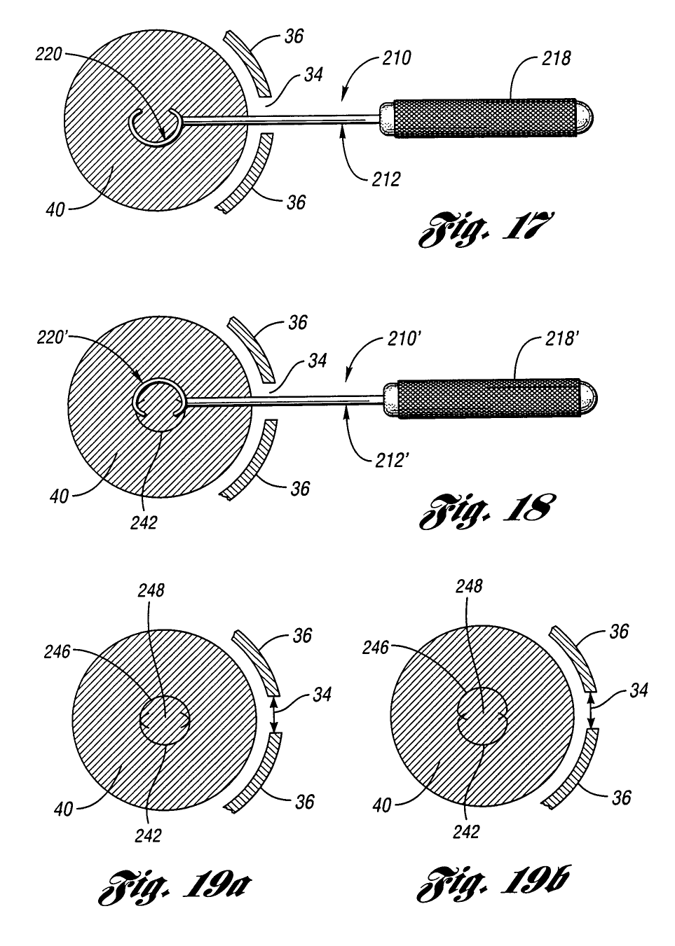

With reference to FIGS. 16a and 16b, yet another alternative instrument 310, 310′ can be provided, wherein reference numerals for instrument 310, 310′ correspond to those for instrument 10, 10′ except for the addition of a “3” prefix. In this embodiment, head portion 320 includes a first portion 360 having a first radius and a second portion 362 continuous with the first portion 360 and having a second radius different from the first radius. In a preferred embodiment, the first radius is larger than the second radius, with the first radius between about 1.0 to 4.0 mm, and the second radius between about 0.25 to 2.0 mm. As shown, first portion 360 is preferably slightly longer than a semicircle, and second portion 362 may have closer to a quarter circle configuration. The cutting edge for instrument 310 is continuous between first and second portions 360, 362, and can include configurations as described above with reference to FIGS. 13a and 13b. In the embodiment depicted herein, handle 312 is connected to head portion 320 at an end 364 of second portion 362. Alternatively, handle 312 can be connected to head portion 320 at an intermediate point of second portion 362 or first portion 360. Instrument 310 could also include at least one third portion, similar to proximal and distal legs 256, 258 (see FIGS. 12a and 12b) having a third radius different from the first and second radii. Head portions 320, 320′ provide an incision with an enlarged area proximal to the wound in order to facilitate access to the lens and cortex as described below with reference to FIGS. 20-22.

As described above, before an incision can be made on the anterior lens capsule, a small entry wound must be cut on the corneal or scleral tissue of the eye, typically using a keratome, in order to gain access to the anterior chamber. It is desirable to have a small corneal/scleral wound for purposes of promoting wound stability, preventing aqueous leakage, and obviating the need for wound suture. Therefore, the wound length should be less than 4 mm, and most preferably on the order of about 2.5 to 3 to mm in length. However, the diameter of the anterior capsule incision usually must be about 4.5 to 5 mm for proper removal of the natural lens and implantation of an artificial intraocular lens. Due to the open curvilinear, preferably semicircular, design of head portion 20, instrument 10 of the present invention is able to accommodate both of these requirements. In fact, instrument 10 could accommodate even smaller entry wounds, since head portion 20 may be eased through the wound using a curved motion, such that the widest corneal/scleral wound needed would be only slightly larger than the width of head portion 20 itself.

Referring now to FIGS. 7-11, the capsulotomy procedure for the eye 32 is initiated by the creation of an entry wound 34 in the corneal or scleral tissue 36 in the manner described above. In addition, a paracentesis (not shown) may be created in order to insert additional instruments into the anterior chamber 38, to create a postoperative entrance, or to inject viscoelastic material into the anterior chamber 38. Injecting the anterior chamber 38 with viscoelastic fluid fully expands the chamber 38 prior to use of instrument 10, and can be performed to avoid any tissue damage as head portion 20 is inserted into position for cutting.

As depicted in FIGS. 7-9, head portion 20 of instrument 10 is inserted through the corneal/scleral wound 34, with cutting edge 24 facing down towards the surface of the anterior capsule 40. If tongue 231 is provided on head portion 20 (see FIG. 15), it is introduced first through wound 34 and holds wound 34 open to facilitate entrance of the instrument 10 into the eye. Introduction of instrument 10 into the eye is viewed and monitored by the operator through a microscope, wherein manipulation of instrument 10 requires only one hand. Next, the anterior capsule 40 is engaged with the cutting edge 24 at a desired location, and sufficient pressure is applied to penetrate at least partially through the anterior capsule 40 in order to create a first generally semicircular incision therein 42. The process of engaging and penetrating the anterior capsule 40 may include a slight rotary motion, such that the semicircular incision extends beyond 180°. In fact, cutting edge 24 can be used to completely penetrate the anterior capsule 40 and even cut the lens 44 with no adverse consequences since the lens 44 will be removed during a subsequent procedure. If desired, a non-toxic dye may be applied to cutting edge 24, such that as cutting edge 24 engages the anterior capsule 40, the dye is deposited and leaves a visual imprint along the boundary of the incision 42.

After the first incision 42 is made, instrument 10 is removed through the wound 34, and instrument 10′ is inserted therethrough and brought into proximity with the anterior capsule 40. As shown in FIG. 10, the orientation of head portion 20′ opposes the orientation of head portion 20 used to make the first incision 42. The anterior capsule 40 is then engaged with cutting edge 24′ with sufficient pressure to penetrate at least partially through the anterior capsule 40 in order to create a second generally semicircular incision 46 therein adjacent the first incision 42. The first and second incisions 42, 46 combine to produce a continuous, generally circular tissue flap 48 incised in the anterior capsule 40 (FIG. 11). The tissue flap 48 is subsequently grasped with forceps or the like and pulled along the incisions 42, 46 in order to completely detach the flap 48 from the anterior capsule 40. Although it is described that incision 42 with a right-facing orientation is performed prior to incision 46 having a left-facing orientation, it is understood that the incisions can be performed in any order to create the continuous tissue flap 48.

Alternatively, instrument 110, wherein head portion 120 has two opposed cutting edges 124, 128, can be used to generate the circular tissue flap 48. The anterior capsule 40 is first engaged with cutting edge 124, which penetrates at least partially through the anterior capsule 40 in order to create a first generally semicircular incision therein 42. Next, instrument 110 is rotated approximately 180° about its longitudinal handle axis 130, after which the anterior capsule 40 is engaged with cutting edge 128 in order to penetrate at least partially through the anterior capsule 40, thereby creating a second generally semicircular incision 46 therein adjacent the first incision 42. As with the combination of instruments 10 and 10′, use of instrument 110 in this manner results in a generally circular tissue flap 48 incised in the anterior capsule 40.

As depicted in FIGS. 17-19, a similar method is followed when using instruments 210 and 210′. Head portion 220 is inserted through the corneal/scleral wound 34, with cutting edge 224 facing down towards the surface of the anterior capsule 40. Next, the anterior capsule 40 is engaged with the cutting edge 224 at a desired location, and sufficient pressure is applied to penetrate at least partially through the anterior capsule 40 in order to create a first incision therein 242. The process of engaging and penetrating the anterior capsule 40 may include a slight rotary motion. After the first incision 242 is made, instrument 210 is removed through the wound 34, and instrument 210′ is inserted therethrough and brought into proximity with the anterior capsule 40. As shown in FIG. 18, the orientation of head portion 220′ opposes the orientation of head portion 220 used to make the first incision 242 (see FIG. 17). The anterior capsule 40 is then engaged with cutting edge 224′ with sufficient pressure to penetrate at least partially through the anterior capsule 40 in order to create a second incision 246 therein adjacent the first incision 242. The first and second incisions 242, 246 combine to produce a continuous tissue flap 248 incised in the anterior capsule 40 (FIG. 19a).

The tissue flap 248 is subsequently grasped with forceps or the like and pulled along the incisions 242, 46 in order to completely detach the flap 248 from the anterior capsule 40. Although it is described that incision 242 with a right-facing orientation is performed prior to incision 246 having a left-facing orientation, it is understood that the incisions can be performed in any order to create the continuous tissue flap 248. Advantageously, a continuous tissue flap is produced using instruments 210, 210′ even when the first and second incisions 242, 246 are not precisely aligned (horizontally, vertically, or rotationally), while still keeping the overall dimensions of the instrument 210 small relative to prior art devices. Furthermore, the inward placement of handle distal end 216 allows the user to align the handle relative to the corneal/scleral incision wound edges or to its center, providing a guide for placement of the two incisions.

Using instruments 310 and 310′, head portion 320 is inserted through the corneal/scleral wound 34, with its cutting edge facing down towards the surface of the anterior capsule 40 (FIG. 20). Manipulation of instrument 310 requires only one hand. Next, the anterior capsule 40 is engaged with the cutting edge at a desired location, and sufficient pressure is applied to penetrate at least partially through the anterior capsule 40 in order to create a first incision therein 342. The process of engaging and penetrating the anterior capsule 40 may include a slight rotary motion. After the first incision 342 is made, instrument 310 is removed through the wound 34, and instrument 310′ is inserted therethrough and brought into proximity with the anterior capsule 40 (FIG. 21), wherein the orientation of head portion 320′ opposes the orientation of head portion 320 used to make the first incision 342. The anterior capsule 40 is then engaged with the cutting edge with sufficient pressure to penetrate at least partially through the anterior capsule 40 in order to create a second generally semicircular incision 346 therein adjacent the first incision 342.

The first and second incisions 342, 346 combine to produce a continuous tissue flap 348 incised in the anterior capsule 40 (FIG. 22). The tissue flap 348 is subsequently grasped with forceps or the like and pulled along the incisions 342, 346 in order to completely detach the flap 348 from the anterior capsule 40. Although it is described that incision 342 with a right-facing orientation is performed prior to incision 346 having a left-facing orientation, it is understood that the incisions can be performed in any order to create the continuous tissue flap 348. Advantageously, the incisions created by instruments 310, 310′ can create an enlarged end of the tissue flap proximal to the wound.

With the tissue flap 48 removed from the anterior capsule 40, phacoemulsification and aspiration procedures or the like can be used to remove the cataractous lens 44, leaving the posterior lens capsule 50 intact to receive the artificial intraocular lens. Ultrasonic energy is used to break up and emulsify the lens nucleus and cortex, a process during which there is a great deal of tension on the cut edges of the anterior capsule 40. The incision created using instrument 10 of the present invention is able to withstand this tension, and prevent tears of the anterior capsule 40 toward the posterior side 50. Once the lens material 44 is completely removed, the artificial intraocular lens (not shown) can be inserted using forceps or other insertion device.

Therefore, instruments of the present invention provide a smooth cut on one side of the anterior capsule, and then on the other, to form a continuous, tissue flap without tears or tags. Furthermore, the instruments allow for a controlled capsulotomy while requiring only a small corneal or scleral wound. The location of the capsular opening is controlled by the surgeon, and the size of the opening is determined by the dimensions of head portion 20. The instruments are easy to operate and offer a simple and efficient surgery with repeatable results, thereby decreasing cost as well as possibility of intraoperative or postoperative complications. Furthermore, the design of the instruments of the present invention makes them simple and cost-effective to manufacture.

Although the instruments and the methods for their use have been described herein in the context of incising the anterior capsule during a capsulotomy procedure, it should be understood that the instruments can be utilized for any surgical procedure where incision of an ocular tissue, or perhaps even other types of tissue, is necessary. For example, an incision similar to that described herein may be required in the posterior capsule during pediatric cataract surgeries. In addition, it is fully contemplated that head portion 20 can have any shape capable of creating an incision suitable for the intended purpose, while still being capable of satisfying the applicable size constraints. Furthermore, it is understood that the numerical values described herein are provided for illustrative purposes only and are typical only for normal situations in cataract surgery. Under other circumstances, different numerical values may apply, and the instruments of the present invention can be adapted accordingly.

While embodiments of the invention have been illustrated and described, it is not intended that these embodiments illustrate and describe all possible forms of the invention. Rather, the words used in the specification are words of description rather than limitation, and it is understood that various changes may be made without departing from the spirit and scope of the invention.

What is claimed is:

1. An instrument for incising an intraocular tissue, comprising:
a handle; and
a head portion attached to the handle and having a cutting edge, wherein the head portion defines a plane and the cutting edge extends from the head portion in a direction substantially transverse to the plane, the head portion including a main portion having a first radius and at least one extension portion continuous with the main portion and having a second radius different from the first radius, the head portion including spaced first and second ends which define an opening therebetween,
wherein the cutting edge is operable to engage the intraocular tissue and penetrate at least partially therethrough to create an incision in the intraocular tissue.

2. The instrument according to claim 1, wherein the second radius is smaller than the first radius.

3. The instrument according to claim 2, wherein the first radius is between about 1.0 to 4.0 mm, and the second radius is between about 0.25 to 2.0 mm.

4. The instrument according to claim 1, wherein the handle is connected to the head portion at an end of the extension portion.

5. The instrument according to claim 1, wherein the handle is connected to the head portion at an intermediate point of the extension portion.

6. The instrument according to claim 1, wherein the handle is connected to the head portion at an intermediate point of the main portion.

7. The instrument according to claim 1, wherein the handle is connected to the head portion at a junction of the main portion and the at least one extension portion.

8. The instrument according to claim 1, wherein the head portion is generally C-shaped and the at least one extension portion comprises a proximal leg extending from a first end of the main portion and a distal leg extending from a second end of the main portion.

9. The instrument according to claim 1, wherein the head portion includes a second cutting edge opposing the cutting edge.

10. The instrument according to claim 1, wherein the head portion has a wavy configuration.

11. The instrument according to claim 1, wherein the cutting edge is serrated.

12. The instrument according to claim 1, wherein the handle includes a generally horizontal portion extending along the plane of the head portion and an angled portion extending at an angle relative to the horizontal portion.

13. The instrument according to claim 1, wherein the handle is connected to the head portion at a location greater than about 0.3 mm above the cutting edge.

14. The instrument according to claim 1, wherein the head portion further includes a tongue extending outwardly from a distal end of the head portion along the plane of the head portion.

15. An instrument for incising an intraocular tissue, comprising:
a handle; and
a generally C-shaped head portion having a cutting edge, wherein the head portion defines a plane and the cutting edge extends from the head portion in a direction substantially transverse to the plane, the head portion including
a generally semicircular central portion having a first radius, the central portion having first and second ends, and
proximal and distal legs continuous with the first and second ends of the central portion, respectively, the legs each having a second radius different from the first radius,
wherein the cutting edge is operable to engage the intraocular tissue and penetrate at least partially therethrough to create a curvilinear incision in the intraocular tissue.

16. The instrument according to claim 15, wherein the second radius is smaller than the first radius.

17. The instrument according to claim 16, wherein the first radius is between about 1.0 to 4.0 mm, and the second radius is between about 0.25 to 2.0 mm.

18. The instrument according to claim 15, wherein the handle is connected to the head portion at a location inward from an end of the proximal leg.

19. An instrument for incising an intraocular tissue, comprising:
a handle; and
a head portion having a cutting edge, wherein the head portion defines a plane and the cutting edge extends from the head portion in a direction substantially transverse to the plane, the head portion including a first portion having a first radius and a second portion continuous with the first portion and having a second radius different from the first radius,
wherein the cutting edge is operable to engage the intraocular tissue and penetrate at least partially therethrough to create an incision in the intraocular tissue.

20. The instrument according to claim 19, wherein the second radius is smaller than the first radius.

21. The instrument according to claim 20, wherein the first radius is between about 1.0 to 4.0 mm, and the second radius is between about 0.25 to 2.0 mm.

22. The instrument according to claim 19, wherein the handle is connected to the head portion at an end of the second portion.

23. The instrument according to claim 19, wherein the handle is connected to the head portion at an intermediate point of one of the first portion and the second portion.

24. A surgical kit for incising an intraocular tissue, comprising:
a first instrument including a handle and a head portion attached to the handle, the first instrument head portion including a main portion having a first radius and at least one extension portion continuous with the main portion and having a second radius different from the first radius, the first instrument head portion having spaced first and second ends which define an opening therebetween having a first orientation, wherein the first instrument head portion includes a cutting edge such that the head portion is operable to engage the intraocular tissue and penetrate at least partially therethrough to create a first incision in the intraocular tissue; and
a second instrument including a handle and a head portion attached to the handle, the second instrument head portion including a main portion having a first radius and at least one extension portion continuous with the main portion and having a second radius different from the first radius, the second instrument head portion having spaced first and second ends which define an opening therebetween having a second orientation, wherein the second instrument head portion includes a cutting edge such that the head portion is operable to engage the intraocular tissue and penetrate at least partially therethrough to create a second incision in the intraocular tissue,
wherein creating adjacent first and second incisions results in a continuous tissue flap incised in the intraocular.

25. The surgical kit according to claim 24, wherein the first and second instruments each include a generally C-shaped head portion including a generally semicircular central portion having a first radius, the central portion having first and second ends, and proximal and distal legs continuous with the first and second ends of the central portion, respectively, the legs each having a second radius smaller than the first radius.

26. The surgical kit according to claim 24, wherein the first and second instruments each include a head portion including a first portion having a first radius and a second portion continuous with the first portion and having a second radius smaller than the first radius.

27. A method for incising an intraocular tissue, comprising:
creating a wound in one of a corneal region and a scleral region of an eye;
inserting an instrument through the wound and into proximity with the intraocular tissue, the instrument including a handle and a head portion attached to the handle having a cutting edge, the head portion including a main portion having a first radius and at least one extension portion continuous with the main portion and having a second radius different from the first radius, the head portion including spaced first and second ends which define an opening therebetween; and
engaging the intraocular tissue with the cutting edge and penetrating at least partially through the intraocular tissue in order to create an incision therein.

28. A method for incising an intraocular tissue, comprising:
creating a wound in one of a corneal region and a scleral region of the eye;
inserting a first instrument through the wound and into proximity with the intraocular tissue, the first instrument including a handle and a head portion attached to the handle, the head portion including a main portion having a first radius and at least one extension portion continuous with the main portion and having a second radius different from the first radius, the first instrument head portion including a cutting edge having a first orientation;
engaging the intraocular tissue with the cutting edge of the first instrument head portion and penetrating at least partially through the intraocular tissue in order to create a first incision therein;
inserting a second instrument through the wound and into proximity with the intraocular tissue, the second instrument including a handle and a head portion attached to the handle, the head portion including a main portion having a first radius and at least one extension portion continuous with the main portion and having a second radius different from the first radius, the second instrument head portion including a cutting edge having a second orientation which opposes the first orientation; and
engaging the intraocular tissue with the cutting edge of the second instrument head portion and penetrating at least partially through the intraocular tissue in order to create a second incision therein adjacent the first incision, thereby resulting in a continuous tissue flap incised in the intraocular tissue.

29. The method according to claim 28, wherein the first and second instruments each include a generally C-shaped head portion including a generally semicircular central portion having a first radius, the central portion having first and second ends, and proximal and distal legs continuous with the first and second ends of the central portion, respectively, the legs each having a second radius smaller than the first radius, wherein imprecise alignment of the first and second incisions can still result in an overlap of the first and second incisions and a continuous tissue flap.

30. The method according to claim 28, wherein the first and second instruments each include a head portion including a first portion having a first radius and a second portion continuous with the first portion and having a second radius smaller than the first radius, wherein the first and second incisions create an enlarged end of the flap proximal to the wound.

31. A method for incising an intraocular tissue, comprising:
creating a wound in one of a corneal region and a scleral region of the eye;
inserting an instrument through the wound and into proximity with the intraocular tissue, the instrument including a handle and a head portion attached to the handle, the head portion having first and second opposed cutting edges;
engaging the intraocular tissue with the first cutting edge and penetrating at least partially through the intraocular tissue in order to create a first incision therein;
rotating the instrument approximately 180° about a longitudinal axis of the handle;
engaging the intraocular tissue with the second cutting edge and penetrating at least partially through the intraocular tissue in order to create a second incision therein adjacent the first incision, thereby resulting in a continuous tissue flap incised in the intraocular tissue.

32. An instrument for incising an intraocular tissue, comprising:
a handle;
a curvilinear head portion attached to the handle and having a cutting edge, wherein the head portion defines a plane and the cutting edge extends from the head portion in a direction substantially transverse to the plane, the head portion including spaced first and second ends which define an opening therebetween; and
a tongue extending outwardly from a distal end of the head portion along the plane of the head portion,
wherein the cutting edge is operable to engage the intraocular tissue and penetrate at least partially therethrough to create an incision in the intraocular tissue.
